(19) United States
(12) Patent Application Publication
(10) Pub. No.: US 2016/0195535 A1
(43) Pub. Date: Jul. 7, 2016

(54) METHOD FOR DETECTING PROSTATIC BASAL CELLS

(71) Applicants: NATIONAL UNIVERSITY CORPORATION HOKKAIDO UNIVERSITY, Hokkaido (JP); LSIP, LLC, Tokyo (JP)

(72) Inventors: Shigetsugu Hatakeyama, Hokkaido (JP); Shinya Tanaka, Hokkaido (JP)

(21) Appl. No.: 14/653,562

(22) PCT Filed: Dec. 18, 2013
(86) PCT No.: PCT/JP2013/083915

§ 371 Date: Jun. 18, 2015

(30) Foreign Application Priority Data
Dec. 20, 2012 (JP) 2012-277980

(51) Int. Cl. G01N 33/574; C07K 16/18

(52) U.S. Cl. CPC G01N 33/57434; C07K 16/18; G01N 2333/4703; G01N 2333/4742

(57) ABSTRACT

The purpose of the present invention is to provide a method for detecting prostatic basal cells by searching for molecules that are expressed specifically in prostatic basal cells and analyzing said molecules. The problem can be solved using a method for detecting prostatic basal cells that is characterized by using immunohistochemical staining to visualize the expression of TRIM29 (Tripartite motif-containing protein 29) protein in prostatic basal cells.

### TECHNICAL FIELD

The present invention relates to a method for detecting prostatic basal cells, a method for identifying the presence, decrease, and disappearance of prostatic basal cells, and an immunohistostaining kit for detecting prostatic basal cells. The present invention enables a reliable detection of prostatic basal cells. The present invention also enables a reliable definite diagnosis of prostate cancer.

### BACKGROUND ART

The normal prostate consists of glandular cells and basal cells, and a histological picture is observed, in which the basal cells are present around the glandular cells.

Androgen receptors, cytokeratin 8, and cytokeratin 18 are specifically expressed in the glandular cells, and most prostate cancers express androgen receptors, cytokeratin 8, or cytokeratin 18 as markers for these glandular cells. Thus, prostate cancer is considered to be derived from the glandular cells.

The basal cells are known to disappear in prostate cancer. Thus, a definite diagnosis of prostate cancer is made by performing the immunohistostaining of prostate using an antibody to p63 or cytokeratin expressed in normal basal cells and identifying the disappearance of the basal cells.

### CITATION LIST

### Patent Literature

Patent Literature 1: International Publication No. WO 2008/150512

Patent Literature 2: International Publication No. WO 2005/014781

Patent Literature 3: International Publication No. WO 2004/018711

Patent Literature 4: International Publication No. WO 2006/080597

Patent Literature 5: International Publication No. WO 2004/018711

### SUMMARY OF INVENTION

### Technical Problem

Regarding the above immunohistostaining of basal cells, it is sometimes difficult to determine whether the basal cells have disappeared, only by immunohistostaining using an anti-p63 antibody or anti-cytokeratin antibody.

Thus, an object of the present invention is to provide a method for detecting prostatic basal cells by searching for molecules specifically expressed in prostatic basal cells and analyzing the molecules.

### Solution to Problem

As a result of intensive studies on molecules specifically expressed in basal cells of normal prostate tissue, the present inventors have surprisingly found that tripartite motif-containing protein 29 (TRIM29) is specifically expressed in the basal cells of normal prostate tissue. Then, it has been shown that the basal cells can be detected by visualizing the expression of TRIM29 in the prostate tissue by means of immunohistostaining.

The present invention is based on these findings.

Specifically, the present invention relates to:

[1] A method for detecting prostatic basal cells, comprising visualizing the expression of tripartite motif-containing protein 29 (TRIM29) protein in basal cells of the prostate by immunohistostaining;
[2] The method for detecting prostatic basal cells according to [1], comprising analyzing the morphology of the glandular tissue structure of the prostate and the visualized expression of tripartite motif-containing protein 29 (TRIM29) protein;
[3] The method for detecting prostatic basal cells according to [1] or [2], further comprising visualizing the expression of cytokeratin and/or p63 protein by immunohistostaining;
[4] The method for detecting prostatic basal cells according to [3], comprising analyzing the morphology of the glandular tissue structure of the prostate and the visualized expression of cytokeratin protein and/or the visualized expression of p63 protein;
[5] A method for identifying the presence, decrease, or disappearance of prostatic basal cells, comprising using the method for detecting prostatic basal cells according to any one of [1] to [4] above;
[6] A method for diagnosing prostate cancer, comprising a step of detecting prostatic basal cells in prostate tissue collected from a subject by immunohistostaining using an anti-TRIM29 antibody or an antigen-binding fragment thereof;
[7] The diagnostic method according to [6], wherein the decrease or disappearance of prostatic basal cells as compared to those in normal prostate tissue indicates the presence of prostate cancer;
[8] An anti-TRIM29 antibody or an antigen-binding fragment thereof for use in the diagnosis of prostate cancer;
[9] A diagnostic composition for diagnosing prostate cancer, comprising an anti-TRIM29 antibody or an antigen-binding fragment thereof;
[10] Use of an anti-TRIM29 antibody or an antigen-binding fragment thereof in the manufacture of a diagnostic composition for diagnosing prostate cancer;
[11] An immunohistostaining kit for detecting prostatic basal cells, comprising an anti-TRIM29 antibody or an antigen-binding fragment thereof; and
[12] The immunohistostaining kit for detecting prostatic basal cells according to [11], further comprising an anti-cytokeratin antibody or an antigen-binding fragment thereof and/or an anti-p63 antibody or an antigen-binding fragment thereof.

The gene of TRIM29 is reported to be associated with lung cancer, ovarian serous papillary adenocarcinoma, and cervical intraepithelial neoplasia (Patent Literatures 1 to 4). A method for predicting prognosis for prostate cancer by examining 80 mRNAs comprising TRIM29 is also disclosed (Patent Literature 5). However, it is not reported that TRIM29 is expressed in basal cells of the normal prostate.

### Advantageous Effects of Invention

The method for detecting prostatic basal cells according to the present invention can reliably detect normal prostatic basal cells and is useful for histological studies of the prostate. The combination of the method with the immunohistostaining of expressed cytokeratin and/or p63 can more reliably detect prostatic basal cells.

The method for detecting prostatic basal cells according to the present invention and the method for identifying the presence, decrease, or disappearance of prostatic basal cells according to the present invention enable a definite diagnosis of prostate cancer. The combination of the methods with the immunohistostaining of expressed cytokeratin and/or p63 enables a more reliable definite diagnosis of prostate cancer.

### BRIEF DESCRIPTION OF DRAWINGS

FIG. 1 is a series of micrographs, each of which shows normal prostate tissue or prostate cancer tissue subjected to the immunohistostaining with anti-TRIM29 polyclonal antibodies or an anti-cytokeratin monoclonal antibody (34βE12).

FIG. 2 is a pair of micrographs, each of which shows normal prostate tissue subjected to the immunohistostaining with an anti-TRIM29 monoclonal antibody.

FIG. 3 is a pair of micrographs, each of which shows normal prostate tissue subjected to the immunohistostaining with an anti-TRIM29 monoclonal antibody or an anti-cytokeratin monoclonal antibody (34βE12).

### DESCRIPTION OF EMBODIMENTS

### [1] Method for Detecting Prostatic Basal Cells

The method for detecting prostatic basal cells according to the present invention comprises visualizing the expression of tripartite motif-containing protein 29 (TRIM29) protein in basal cells of the prostate by immunohistostaining. According to the method, prostate-derived cells expressing TRIM29 can be determined to be basal cells based on their morphology and protein expression. In addition, the method enables the detection of basal cells which form part of the glandular tissue structure by identifying the morphology of the glandular tissue structure of the prostate and the visualized expression of TRIM29 protein.

The animal species having prostatic basal cells which may be detected by the detection method of the present invention is not limited as long as it has basal cells in the prostate; and examples of the animal species include mammals, such as humans, monkeys, dogs, cats, ferrets, cows, horses, goats, sheep, guinea pigs, hamsters, jirds, mice, or rats. The detection method of the present invention can also be used in separated cells. For example, the method can also be applied to primary cultured cells or passage cells comprising separated basal cells from any of the above mammals.

### <Prostate>

Prostate is a gland in the male reproductive system and is located beneath the bladder and in front of the rectum. The prostate is composed of two types of cells: basal cells and glandular cells. Histologically, prostatic basal cells are present around prostatic glandular cells.

### (Prostatic Glandular Cells)

Prostatic glandular cells specifically express androgen receptors, cytokeratin 8, and cytokeratin 18, and most prostate cancers are derived from prostatic glandular cells.

### (Prostatic Basal Cells)

The prostatic basal cells which may be detected by the detection method of the present invention are not limited as long as the cells express TRIM29. However, this does not exclude the presence of prostatic basal cells not expressing TRIM29.

The basal cells specifically express p63, cytokeratin 5, and cytokeratin 14 in addition to TRIM29. p63 is considered to play an important role in the development of the prostate; the nuclei of the basal cells are stained by immunohistostaining with 4A4 which is an anti-p63 monoclonal antibody. Cytokeratin 5 and cytokeratin 14 are present in the cytoplasm and stained by immunohistostaining with 34βE12 which is an anti-cytokeratin monoclonal antibody. The monoclonal antibody 34βE12 is an antibody exhibiting positive reactions to cytokeratin 1 and cytokeratin 10 in addition to cytokeratin 5 and cytokeratin 14.

### <TRIM29>

Tripartite motif-containing protein 29 (TRIM29) belongs to the TRIM gene family, and is also known as ATDC (ataxia-telangiectasia group D-associated protein). Human TRIM29 consists of 588 amino acids. TRIM29 has a plurality of zinc finger motifs and a leucine zipper motif and birds to DNA. TRIM29 is considered to possibly function as a transcriptional regulator in differentiation processes. TRIM29 is known to be associated with ataxia-telangiectasia, and has been pointed out to be associated with the function of suppressing radiation sensitivity.

According to the present invention, the base sequence of a nucleic acid encoding analyzable human TRIM29 protein (hereinafter also referred to as human TRIM29 gene) is shown in SEQ ID NO: 1, and the amino acid sequence of the human TRIM29 protein is shown in SEQ ID NO: 2. However, the gene to be analyzed is not limited to the TRIM29 gene having the base sequence as shown in SEQ ID NO: 1 as long as it is expressed in prostatic basal cells and can hybridize to a probe or a primer capable of binding to the TRIM29 gene having the base sequence of SEQ ID NO: 1. The protein to be analyzed is also not limited to the TRIM29 protein having the amino acid sequence as shown in SEQ ID NO: 2 as long as it is expressed in prostatic basal cells and can bind to an antibody capable of binding to the protein having the amino acid sequence of SEQ ID NO: 2. In other words, according to the present invention, the TRIM29 to be analyzed may be TRIM29 having a mutation or mutations as long as it is expressed in prostatic basal cells. It is also not limited to human TRIM29 and may be mammalian TRIM29 including, for example, the TRIM29 of humans, monkeys, dogs, cats, ferrets, cows, horses, goats, sheep, guinea pigs, hamsters, jirds, mice, or rats. Examples of TRIM29 protein having a mutation or mutations include, when human TRIM29 is taken for instance, a protein consisting of an amino acid sequence in which 1 to several, e.g., 1, 2, 3, 4, 5, 6, 7, 8, or 9, amino acids are substituted, deleted, inserted, or added in the amino acid sequence of SEQ ID NO: 2, and a protein consisting of an amino acid sequence having 80% or more sequence identity, e.g., 80% or more, 85% or more, 90% or more, 95% or more, or 98% or more sequence identity, with the amino acid sequence of SEQ ID NO: 2.

### <TRIM29 Protein Expression Analysis>

The TRIM29 expression analysis by immunohistostaining according to the detection method of the present invention involves, but is not limited to, visualizing the expression of TRIM29 protein. By the visualization of the expression of TRIM29 protein, prostatic basal cells can be morphologically identified in the same basal cells, and the morphologies of prostatic basal cells and the glandular tissue structure of the prostate can be identified based on the expression of TRIM29 protein. In other words, the detection method of the present invention preferably involves analyzing, in basal cells which form part of the glandular tissue structure, the morphology of the glandular tissue structure of the prostate and the visualized expression of TRIM29 protein.

As used herein, the “visualization” does not only mean the gross identification of coloring or fluorescence resulting from immunohistostaining through a light microscope or a fluorescence microscope, but includes, for example, the mechanical detection of coloring, fluorescence, luminescence, or radiation (i.e., a signal) followed by the identification of a picture or the like obtained by imaging the signal.

As used herein, the “analyzing the morphology of the glandular tissue structure of the prostate and the visualized expression of the tripartite motif-containing protein 29 (TRIM29) protein” includes not only the simultaneous observation of the morphology of the glandular tissue structure of the prostate and the expression of TRIM29 protein, but also, for example, the observation of the morphology of the glandular tissue structure of the prostate in comparison with the picture or the like obtained by imaging the signal.

The analysis of TRIM29 protein in prostatic basal cells is carried out by immunohistostaining. This is because the position of basal cells in the prostate tissue or the morphology of basal cells can be identified by immunohistostaining.

### (Immunohistostaining)

Immunohistostaining is an established technique and can be carried out based on a known method except for the use of an antibody specific for TRIM29. In other words, immunohistostaining is a method which involves detecting a specific antigen expressed in cells of tissue slices by using an antibody specifically recognizing the antigen.

Specifically, immunohistostaining can be carried out, for example, as follows. A prostate tissue is frozen and sliced, or a fixed, paraffin-embedded tissue block is sliced to prepare tissue slices. An antibody recognizing TRIM29 is reacted with the surface of the tissue slice. The anti-TRIM29 antibody can be labeled with a substance emitting a signal to detect TRIM29 protein on each tissue slice. Alternatively, a second antibody to the anti-TRIM29 antibody may be labeled with a substance emitting a signal without labeling the anti-TRIM29 antibody. In addition, biotin may be bound to the anti-TRIM29 antibody or the second antibody, followed by labeling avidin, specifically binding to biotin, with a substance emitting a signal.

The substance which may be used for labeling the antibody includes, but is not limited to, a fluorescent dye (e.g., rhodamine, fluorescein isothiocyanate (FITC), or a rare earth metal chelate), a radioactive substance (e.g., ³H, ¹⁴C, or ¹²⁵I), or an enzyme (e.g., peroxidase, alkaline phosphatase, or β-D-galactosidase). The detection of a signal can be carried out using, but not limited to, fluorescence, radiation (autoradiography), luminescence, and coloring. For example, when rhodamine, or FITC is used as the fluorescent dye, the signal can be detected using a fluorescence microscope. For example, when alkaline phosphatase is used and coloring is performed using NBT/BCIP, the signal can be detected under a light microscope and the morphology of cells can simultaneously be observed.

Specifically, commonly used methods include a peroxidase anti-peroxidase method (PAP method), a streptavidin-biotin complex method (sABC method), a polymer reagent method in which a secondary antibody and a labeling enzyme are bound to a polymer, and a method using a primary antibody labeled with FITC or using an HRP-labeled anti-FITC antibody as a secondary antibody.

### (Anti-TRIM29 Antibody)

The anti-TRIM29 antibody used for the immunohistostaining according to the present invention can be prepared by a known method except for using TRIM29 protein or a partial peptide thereof as an immunogen. The anti-TRIM29 antibody may be polyclonal antibodies or a monoclonal antibody; and a monoclonal antibody is preferable. This is because a monoclonal antibody often produces less cross-reaction with other proteins. A known or commercially available antibody can also be used.

For example, a monoclonal antibody can be prepared according to the method of Koehler and Milstein (Nature 256: 495-497, 1975). For polyclonal antibodies, for example, NKG2D ligand alone or an antigen bound to BSA or KLH can be mixed with an adjuvant, such as Freund's complete adjuvant, and intracutaneously immunized into a rabbit on a periodic basis, followed by collecting blood once the antibody titer in the blood has elevated, and the collected blood can be used as an antiserum or the antibodies can be purified by a known method before use.

The antibody used in the immunological analysis method may also be an antibody fragment containing an antigen-binding site for TRIM29 protein (antigen-binding fragment). Examples of the antibody fragment include F(ab′)₂, Fab′, Fab, and Fv. These antibody fragments can each be obtained, for example, by digesting an antibody with a proteinase (for example, pepsin or papain) by a conventional method and subsequently purifying the resultant by a conventional method for separating and purifying a protein.

In the method for detecting prostatic basal cells according to the present invention, it is preferable to perform the expression analysis of cytokeratin protein and/or p63 protein in addition to the TRIM29 protein expression analysis. In other words, TRIM29 protein and cytokeratin protein may be analyzed; TRIM29 protein and p63 protein may be analyzed; or the three proteins may be analyzed.

According to the present embodiment, 2 or more proteins may be analyzed in the same tissue slice from the same subject (patient). In addition, 2 or more proteins may be separately analyzed in each of different tissue slices from the same subject (patient).

### <Cytokeratin Protein Expression Analysis>

The cytokeratin expression analysis by immunohistostaining in the detection method of the present invention involves, but is not limited to, visualizing the expression of cytokeratin protein. By the visualization of the expression of cytokeratin protein, prostatic basal cells can be morphologically identified in the same basal cells, and the morphologies of prostatic basal cells and the glandular tissue structure of the prostate can be identified based on the expression of cytokeratin protein. In other words, the detection method of the present invention preferably involves analyzing the morphology of the glandular tissue structure of the prostate and the visualized expression of cytokeratin protein in basal cells which form part of the glandular tissue structure.

The expression analysis of cytokeratin protein can be carried out based on a known method except for using an antibody specific for cytokeratin. Specifically, the analysis can be carried out according to the method described in “TRIM29 Protein Expression Analysis” except for using an antibody specific for cytokeratin. The amino acid sequence of cytokeratin 5 is shown in SEQ ID NO: 4, and the amino acid sequence of cytokeratin 14 is shown in SEQ ID NO: 6. The base sequence encoding cytokeratin 5 is shown in SEQ ID NO: 3 and the base sequence encoding cytokeratin 14 is shown in SEQ ID NO: 5.

The antibody specific for cytokeratin may be polyclonal antibodies or a monoclonal antibody; and a monoclonal antibody is preferable. This is because a monoclonal antibody often produces less cross-reaction with other proteins. A known or commercially available antibody can also be used. For example, 34βE12 monoclonal antibody can be used, which is a commercially available anti-cytokeratin monoclonal antibody; this antibody recognizes cytokeratins 1, 5, 10, and 14.

### <p63 Protein Expression Analysis>

The p63 expression analysis in the detection method of the present invention involves, but is not limited to, visualizing the expression of p63 protein. By the visualization of the expression of p63 protein, prostatic basal cells can be morphologically identified in the same basal cells, and the morphologies of prostatic basal cells and the glandular tissue structure of the prostate can be identified based on the expression of p63 protein. In other words, the detection method of the present invention preferably involves analyzing the morphology of the glandular tissue structure of the prostate and the visualized expression of p63 protein in basal cells which form part of the glandular tissue structure.

The expression analysis of p63 protein can be carried out based on a known method except for using an antibody specific for p63. Specifically, the analysis can be carried out according to the method described in “TRIM29 Protein Expression Analysis” except for using an antibody specific for p63. The amino acid sequence of p63 is shown in SEQ ID NO: 8 and the base sequence encoding p63 is shown in SEQ ID NO: 7.

The antibody specific for p63 may be polyclonal antibodies or a monoclonal antibody; and a monoclonal antibody is preferable. This is because a monoclonal antibody often produces less cross-reaction with other proteins. A known or commercially available antibody can also be used. For example, 4A4 monoclonal antibody can be used, which is a commercially available anti-p63 monoclonal antibody.

### [2] Method for Identifying Presence, Decrease, or Disappearance of Prostatic Basal Cells

The method for identifying the presence, decrease, or disappearance of prostatic basal cells according to the present invention comprises visualizing the expression of tripartite motif-containing protein 29 (TRIM29) protein in the prostate tissue by means of immunohistostaining using the method for detecting prostatic basal cells according to the present invention. In the identification method, the expression of TRIM29 protein is detected. The detection of the expression of TRIM29 protein can be carried out in the same manner as described in “TRIM29 Protein Expression Analysis” in the method for detecting prostatic basal cells according to the present invention. After detecting prostatic basal cells, the presence, decrease, or disappearance of the prostatic basal cells is identified.

In the method for identifying the presence, decrease, or disappearance of prostatic basal cells according to the present invention, the visualization of the expression of TRIM29 protein by immunohistostaining enables the observation of the relation between the morphologies of the basal cells and the glandular tissue structure of the prostate and the expression of TRIM29 protein, enabling the reliable identification of the presence, decrease, or disappearance of prostatic basal cells which form part of the glandular tissue structure.

For example, when the presence, decrease, or disappearance of prostatic basal cells is identified on tissue sections, the “presence,” “decrease,” or “disappearance” of TRIM29 protein in a test sample can be visually determined under a fluorescence microscope or a light microscope based on the visualized expression level of TRIM29 protein as compared to the expression level of TRIM29 protein in the basal cells of the normal prostate tissue. A signal, such as coloring, luminescence, fluorescence, or radiation, in an image taken with a camera or the like can be mechanically visualized to analyze the expression level of TRIM29 protein. Such a method enables the determination of the “presence,” “decrease,” or “disappearance” of TRIM29 protein in a test sample by seeing the image with the naked eye and also enables the determination of the “presence,” “decrease,” or “disappearance” of TRIM29 protein by the mechanical digitization of the signal.

For example, when the level of TRIM29 protein in a test sample is comparable as compared to the expression level of TRIM29 protein in basal cells of the normal prostate tissue, prostatic basal cells can be determined to be “present” in the test sample. When little TRIM29 protein can be detected in a test sample, prostatic basal cells can be determined to “disappear.” When the level of TRIM29 protein in a test sample is intermediate between those for “presence” and “disappearance,” prostatic basal cells can be determined to “decrease.”

### <Prostate Cancer>

In prostate cancer, prostatic basal cells completely disappear, or decrease with some cells remaining. Thus, the method for detecting prostatic basal cells and the method for identifying the presence, decrease, and disappearance of prostatic basal cells according to the present invention enable a diagnosis or definite diagnosis of prostate cancer.

### [3] Immunohistostaining Kit

The immunohistostaining kit for detecting prostatic basal cells according to the present invention comprises an anti-TRIM29 antibody. The immunohistostaining kit of the present invention can be used in the method for detecting prostatic basal cells, the method for identifying the presence, decrease, or disappearance of prostatic basal cells, and a method for diagnosing prostate cancer.

The immunohistostaining kit of the present invention preferably comprises an antibody specifically binding to TRIM29 or a fragment having its antigen-binding site (antigen-binding fragment) in a desired form. The anti-TRIM29 antibody used here may be the anti-TRIM29 antibody described in “[1] Method for Detecting Prostatic Basal Cells.” In other words, the antibody may be a monoclonal antibody or polyclonal antibodies. The antibody fragment is not particularly limited as long as it has the ability to specifically bind to TRIM29, i.e., it is a fragment having the antigen-binding site (antigen-binding fragment). The antibody fragment used here may be, for example, Fab, Fab′, F(ab′)₂, or Fv.

In addition, the immunohistostaining kit for detecting prostatic basal cells according to the present invention preferably comprises an anti-cytokeratin antibody and/or an anti-p63 antibody. The anti-cytokeratin antibody and the anti-p63 antibody used here may also be the anti-cytokeratin antibody and anti-p63 antibody described in “[1] Method for Detecting Prostatic Basal Cells,” or fragments having their antigen-binding sites (antigen-binding fragments).

In addition, the immunohistostaining kit for detecting prostatic basal cells according to the present invention may comprise an enzyme such as peroxidase, alkaline phosphatase, β-D-galactosidase, and glucose oxidase. The kit may also comprise, for example, fluorescein isothiocyanate or a rare earth metal chelate as a fluorescent substance. The kit may also comprise a radioactive isotope such as ³F, ¹⁴C, and ¹²⁵I. The kit according to the present invention may use other labeling substances such as biotin, avidin, and a chemiluminescent substance. The kit of the present invention may comprise, for example, a suitable substrate for the enzyme or chemiluminescent substance.

The kit of the present invention may comprise instructions stating that it is for the detection of prostatic basal cells. The description to the effect that it is for the detection of prostatic basal cells may be presented on a container for the kit. It may be stated that the kit is used for the diagnosis or definite diagnosis of prostate cancer.

The anti-TRIM29 antibody can be used for the manufacture of the immunohistostaining kit for detecting prostatic basal cells. The anti-cytokeratin antibody can also be used for the manufacture of the immunohistostaining kit for detecting prostatic basal cells. In addition, the anti-p63 antibody can be used for the manufacture of the immunohistostaining kit for detecting prostatic basal cells.

### EXAMPLES

The present invention is specifically described below with reference to Examples, which are not intended to limit the scope of the present invention.

### Example 1

In this Example, the immunohistostaining of prostate tissues (normal and cancer) was carried out using an anti-TRIM29 antibody. The immunohistostaining was performed using prostate specimens of 16 samples stored and managed in Department of Cancer Pathology, Hokkaido University Graduate School of Medicine. The profiles of the prostate specimens provided for analysis are shown in Table 1. Fifteen samples having prostate cancer and 1 sample having prostatic intraepithelial neoplasia (PIN) were analyzed.

TABLE 1
Clinicopathologic Findings in 15 Cases having Prostate Cancer

| | n (%) | |
|---|---|---|
| Age (years) | | |
| <65 | 2 | (13.3) |
| 65-75 | 7 | (46.7) |
| >75 | 6 | (40) |
| PSA (ng/ml) | | |
| 4 < PSA < 6 | 5 | (33.3) |
| 6 ≦ PSA < 10 | 8 | (53.3) |
| 10< | 2 | (13.3) |
| T (Extent of Primary Tumor) | | |
| T1c | 12 | (80) |
| T2a | 2 | (13.3) |
| T2b | 0 | (0) |
| T2c | 1 | (6.7) |
| N (Lymph Node Metastasis) | | |
| N0 | 15 | (100.0) |
| N1< | 0 | (0) |
| M (Distant Metastasis) | | |
| M0 | 15 | (100) |
| M1< | 0 | (0) |
| TNM Stage | | |
| I | 0 | (0) |
| II | 15 | (100) |
| III | 0 | (0) |
| IV | 0 | (0) |

(1) Staining with Anti-TRIM29 Rabbit Polyclonal Antibodies

An anti-TRIM29 rabbit polyclonal antibodies (ATDC(H-300)SC-33151 manufactured by Santa Cruz) as a primary antibody were diluted 200 times with a diluent (PBS containing 1% BSA) and added to each tissue slice. The primary reaction was carried out at 37° C. for 30 minutes. The resultant tissue slices were washed 3 times with a wash solution (PBS), and a reagent containing a peroxidase-labeled anti-rabbit antibody (Dako REAL™ EnVision™ Detection Reagent peroxidase rabbit/mouse) as a secondary antibody was added to the tissue slice. The second reaction was carried out at room temperature for 30 minutes. The resultant was washed 3 times with the wash solution and then subjected to coloring reaction using a coloring solution (REAL™ DAB+CHROMOGEN from Dako).

(2) Staining with Anti-Cytokeratin Monoclonal Antibody

In addition, the immunohistostaining of prostate tissues (normal and cancer) was carried out using an anti-cytokeratin monoclonal antibody, 34βE12, as a primary antibody. The steps described in the above (1) were repeated except for using 34βE12 (H1205 Nichirei anti-polymer cytokeratin monoclonal antibody) diluted 2 times with 1% BSA in place of the anti-TRIM29 rabbit polyclonal antibody diluted 200 times.

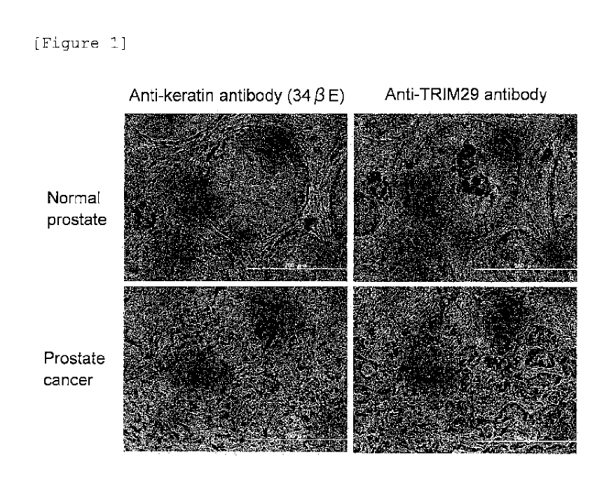

The results of immunohistostaining as described in the above (1) and (2) are shown in FIG. 1. It turned out that the anti-TRIM29 antibody specifically stained basal cells present around glandular cells in the normal prostate tissue. It turned out that cells and tissue stained by the anti-TRIM29 antibody decreased or disappeared in the prostate cancer tissue. These results were comparable to those obtained when immunohistostaining was carried out using 34βE12. The combined use of 34βE12 and the anti-TRIM29 antibody can increase the reliability of the diagnosis of prostate cancer.

### Example 2

In this Example, the immunohistostaining of normal prostate was carried out using an anti-TRIM29 mouse monoclonal antibody (anti-ATDC (A-5) antibody) (sc-166718) as a primary antibody.

The steps described in subsection (1) of Example 1 were repeated except that the anti-TRIM29 mouse monoclonal antibody A-5 was used as a primary antibody, that an anti-mouse IgG antibody was used as a secondary antibody, and that only normal prostate tissue was used as a sample.

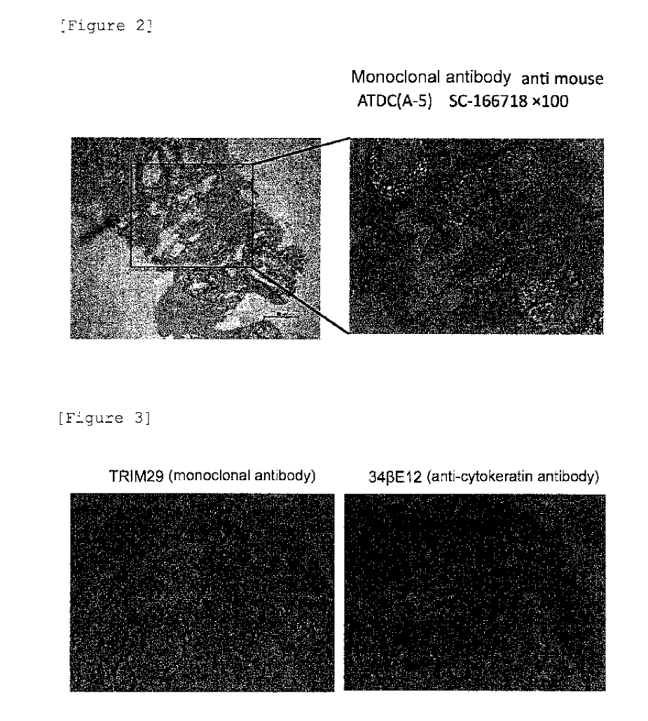

As shown in FIG. 2, non-specific staining was not observed and basal cells were clearly stained.

### Example 3

In this Example, the immunohistostaining of normal prostate was carried out using an anti-TRIM29 mouse monoclonal antibody (anti-ATDC (A-5) antibody) (sc-166718) and an anti-cytokeratin monoclonal antibody.

The steps described in Example 2 were repeated for the anti-TRIM29 mouse monoclonal antibody, and the steps described in subsection (2) of Example 1 were repeated for the anti-cytokeratin monoclonal antibody except for using only normal prostate tissue.

As shown in FIG. 3, the staining with the anti-TRIM29 mouse monoclonal antibody provided a result very similar to that of the staining with the anti-cytokeratin monoclonal antibody; and the combination of these two immunohistostainings enabled the reliable identification of prostatic basal cells.

When the results of immunohistostaining in Examples 1 to 3 were analyzed, the expression of TRIM29 was found to disappear in cancer tissue portions for all cases of prostate cancer. In contrast, for prostatic intraepithelial neoplasia, the expression of TRIM29 was observed in basal cell portions as with the normal prostate. These results are shown in Table 2.

TABLE 2
Degree of TRIM29 Staining in Prostate Cancer

| | Percentage of TRIM29-Positive Acini | | | |
|---|---|---|---|---|
| | in All Acini | | | |
| Gleason Score | 0 | 0-30 | 30-60 | 60-100 |
| Gleason score 3 + 3 = 6 (n = 8) | 8 | 0 | 0 | 0 |
| Gleason score 3 + 4 = 7 (n = 3) | 3 | 0 | 0 | 0 |
| Gleason score 4 + 3 = 7 (n = 1) | 1 | 0 | 0 | 0 |
| Gleason score 4 + 4 = 8 (n = 2) | 2 | 0 | 0 | 0 |
| Gleason score 4 + 5 = 9 (n = 1) | 1 | 0 | 0 | 0 |
| PIN (n = 1) | 0 | 0 | 0 | 1 |
| Normal site (n = 15) | 0 | 0 | 0 | 15 |

### INDUSTRIAL APPLICABILITY

The method for detecting prostatic basal cells according to the present invention can be used for the histological study of prostate. The method for identifying the presence, decrease, or disappearance of prostatic basal cells according to the present invention enables a definite diagnosis of prostate cancer. In addition, the combination of the methods with the expression analysis of cytokeratin enables a more reliable definite diagnosis of prostate cancer.

1. A method for detecting prostatic basal cells, comprising visualizing the expression of tripartite motif-containing protein 29 (TRIM29) protein in basal cells of a prostate by immunohistostaining.

2. The method for detecting prostatic basal cells according to claim 1, comprising analyzing the morphology of the glandular tissue structure of the prostate and the visualized expression of tripartite motif-containing protein 29 (TRIM29) protein.

3. The method for detecting prostatic basal cells according to claim 1, further comprising visualizing the expression of cytokeratin and/or p63 protein by immunohistostaining.

4. The method for detecting prostatic basal cells according to claim 3, comprising analyzing the morphology of the glandular tissue structure of the prostate and the visualized expression of cytokeratin protein and/or the visualized expression of p63 protein.

5. A method for identifying the presence, decrease, or disappearance of prostatic basal cells, comprising using the method for detecting prostatic basal cells according to claim 1.

6. A method for diagnosing prostate cancer, comprising a step of detecting prostatic basal cells in prostate tissue collected from a subject by immunohistostaining using an anti-TRIM29 antibody or an antigen-binding fragment thereof.

7. The diagnostic method according to claim 6, wherein the decrease or disappearance of prostatic basal cells as compared to those in normal prostate tissue indicates the presence of prostate cancer.

8-10. (canceled)

11. An immunohistostaining kit for detecting prostatic basal cells, comprising an anti-TRIM29 antibody or an antigen-binding fragment thereof.

12. The immunohistostaining kit for detecting prostatic basal cells according to claim 11, further comprising an anti-cytokeratin antibody or an antigen-binding fragment thereof and/or an anti-p63 antibody or an antigen-binding fragment thereof.


SEQUENCE LISTING (8 sequences)

SEQ ID NO: 1 - DNA, 1767 bp, Homo sapiens
atggaagctgcagatgcctccaggagcaacgggtcgagcccagaagccagggatgcccggagcccgtcgggccccagtggcagcctggagaatggcaccaaggctgacggcaaggatgccaagaccaccaacgggcacggcggggaggcagctgagggcaagagcctgggcagcgccctgaagccaggggaaggtaggagcgccctgttcgcgggcaatgagtggcggcgacccatcatccagtttgtcgagtccggggacgacaagaactccaactacttcagcatggactctatggaaggcaagaggtcgccgtacgcagggctccagctgggggctgccaagaagccacccgttacctttgccgaaaagggcgagctgcgcaagtccattttctcggagtcccggaagcccacggtgtccatcatggagcccggggagacccggcggaacagctacccccgggccgacacgggccttttttcacggtccaagtccggctccgaggaggtgctgtgcgactcctgcatcggcaacaagcagaaggcggtcaagtcctgcctggtgtgccaggcctccttctgcgagctgcatctcaagccccacctggagggcgccgccttccgagaccaccagctgctcgagcccatccgggactttgaggcccgcaagtgtcccgtgcatggcaagacgatggagctcttctgccagaccgaccagacctgcatctgctacctttgcatgttccaggagcacaagaatcatagcaccgtgacagtggaggaggccaaggccgagaaggagacggagctgtcattgcaaaaggagcagctgcagctcaagatcattgagattgaggatgaagctgagaagtggcagaaggagaaggaccgcatcaagagcttcaccaccaatgagaaggccatcctggagcagaacttccgggacctggtgcgggacctggagaagcaaaaggaggaagtgagggctgcgctggagcagcgggagcaggatgctgtggaccaagtgaaggtgatcatggatgctctggatgagagagccaaggtgctgcatgaggacaagcagacccgggagcagctgcatagcatcagcgactctgtgttgtttctgcaggaatttggtgcattgatgagcaattactctctccccccacccctgcccacctatcatgtcctgctggagggggagggcctgggacagtcactaggcaacttcaaggacgacctgctcaatgtatgcatgcgccacgttgagaagatgtgcaaggcggacctgagccgtaacttcattgagaggaaccacatggagaacggtggtgaccatcgctatgtgaacaactacacgaacagcttcgggggtgagtggagtgcaccggacaccatgaagagatactccatgtacctgacacccaaaggtggggtccggacatcataccagccctcgtctcctggccgcttcaccaaggagaccacccagaagaatttcaacaatctctatggcaccaaaggtaactacacctcccgggtctgggagtactcctccagcattcagaactctgacaatgacctgcccgtcgtccaaggcagctcctccttctccctgaaaggctatccctccctcatgcggagccaaagccccaaggcccagccccagacttggaaatctggcaagcagactatgctgtctcactaccggccattctacgtcaacaaaggcaacgggattgggtccaacgaagccccatga

SEQ ID NO: 2 - protein, 588 aa, Homo sapiens
MEAADASRSNGSSPEARDARSPSGPSGSLENGTKADGKDAKTTNGHGGEAAEGKSLGSALKPGEGRSALFAGNEWRRPIIQFVESGDDKNSNYFSMDSMEGKRSPYAGLQLGAAKKPPVTFAEKGELRKSIFSESRKPTVSIMEPGETRRNSYPRADTGLFSRSKSGSEEVLCDSCIGNKQKAVKSCLVCQASFCELHLKPHLEGAAFRDHQLLEPIRDFEARKCPVHGKTMELFCQTDQTCICYLCMFQEHKNHSTVTVEEAKAEKETELSLQKEQLQLKIIEIEDEAEKWQKEKDRIKSFTTNEKAILEQNFRDLVRDLEKQKEEVRAALEQREQDAVDQVKVIMDALDERAKVLHEDKQTREQLHSISDSVLFLQEFGALMSNYSLPPPLPTYHVLLEGEGLGQSLGNFKDDLLNVCMRHVEKMCKADLSRNFIERNHMENGGDHRYVNNYTNSFGGEWSAPDTMKRYSMYLTPKGGVRTSYQPSSPGRFTKETTQKNFNNLYGTKGNYTSRVWEYSSSIQNSDNDLPVVQGSSSFSLKGYPSLMRSQSPKAQPQTWKSGKQTMLSHYRPFYVNKGNGIGSNEAP

SEQ ID NO: 3 - DNA, 1773 bp, Homo sapiens
atgtctcgccagtcaagtgtgtccttccggagcgggggcagtcgtagcttcagcaccgcctctgccatcaccccgtctgtctcccgcaccagcttcacctccgtgtcccggtccgggggtggcggtggtggtggcttcggcagggtcagccttgcgggtgcttgtggagtgggtggctatggcagccggagcctctacaacctggggggctccaagaggatatccatcagcactagtggtggcagcttcaggaaccggtttggtgctggtgctggaggcggctatggctttggaggtggtgccggtagtggatttggtttcggcggtggagctggtggtggctttgggctcggtggcggagctggctttggaggtggcttcggtggccctggctttcctgtctgccctcctggaggtatccaagaggtcactgtcaaccagagtctcctgactcccctcaacctgcaaatcgaccccagcatccagagggtgaggaccgaggagcgcgagcagatcaagaccctcaacaataagtttgcctccttcatcgacaaggtgcggttcctggagcagcagaacaaggttctggacaccaagtggaccctgctgcaggagcagggcaccaagactgtgaggcagaacctggagccgttgttcgagcagtacatcaacaacctcaggaggcagctggacagcatcgtgggggaacggggccgcctggactcagagctgagaaacatgcaggacctggtggaagacttcaagaacaagtatgaggatgaaatcaacaagcgtaccactgctgagaatgagtttgtgatgctgaagaaggatgtagatgctgcctacatgaacaaggtggagctggaggccaaggttgatgcactgatggatgagattaacttcatgaagatgttctttgatgcggagctgtcccagatgcagacgcatgtctctgacacctcagtggtcctctccatggacaacaaccgcaacctggacctggatagcatcatcgctgaggtcaaggcccagtatgaggagattgccaaccgcagccggacagaagccgagtcctggtatcagaccaagtatgaggagctgcagcagacagctggccggcatggcgatgacctccgcaacaccaagcatgagatctctgagatgaaccggatgatccagaggctgagagccgagattgacaatgtcaagaaacagtgcgccaatctgcagaacgccattgcggatgccgagcagcgtggggagctggccctcaaggatgccaggaacaagctggccgagctggaggaggccctgcagaaggccaagcaggacatggcccggctgctgcgtgagtaccaggagctcatgaacaccaagctggccctggacgtggagatcgccacttaccgcaagctgctggagggcgaggaatgcagactcagtggagaaggagttggaccagtcaacatctctgttgtcacaagcagtgtttcctctggatatggcagtggcagtggctatggcggtggcctcggtggaggtcttggcggcggcctcggtggaggtcttgccggaggtagcagtggaagctactactccagcagcagtgggggtgtcggcctaggtggtgggctcagtgtggggggctctggcttcagtgcaagcagtggccgagggctgggggtgggctttggcagtggcgggggtagcagctccagcgtcaaatttgtctccaccacctcctcctcccggaagagcttcaagagctaa

SEQ ID NO: 4 - protein, 590 aa, Homo sapiens
MSRQSSVSFRSGGSRSFSTASAITPSVSRTSFTSVSRSGGGGGGGFGRVSLAGACGVGGYGSRSLYNLGGSKRISISTSGGSFRNRFGAGAGGGYGFGGGAGSGFGFGGGAGGGFGLGGGAGFGGGFGGPGFPVCPPGGIQEVTVNQSLLTPLNLQIDPSIQRVRTEEREQIKTLNNKFASFIDKVRFLEQQNKVLDTKWTLLQEQGTKTVRQNLEPLFEQYINNLRRQLDSIVGERGRLDSELRNMQDLVEDFKNKYEDEINKRTTAENEFVMLKKDVDAAYMNKVELEAKVDALMDEINFMKMFFDAELSQMQTHVSDTSVVLSMDNNRNLDLDSIIAEVKAQYEEIANRSRTEAESWYQTKYEELQQTAGRHGDDLRNTKHEISEMNRMIQRLRAEIDNVKKQCANLQNAIADAEQRGELALKDARNKLAELEEALQKAKQDMARLLREYQELMNTKLALDVEIATYRKLLEGEECRLSGEGVGPVNISVVTSSVSSGYGSGSGYGGGLGGGLGGGLGGGLAGGSSGSYYSSSSGGVGLGGGLSVGGSGFSASSGRGLGVGFGSGGGSSSSVKFVSTTSSSRKSFKS

SEQ ID NO: 5 - DNA, 1419 bp, Homo sapiens
atgaccacctgcagccgccagttcacctcctccagctccatgaagggctcctgcggcatcgggggcggcatcgggggcggctccagccgcatctcctccgtcctggccggagggtcctgccgcgcccccagcacctacgggggcggcctgtctgtctcatcctcccgcttctcctctgggggagcctacgggctggggggcggctatggcggtggcttcagcagcagcagcagcagctttggtagtggctttgggggaggatatggtggtggccttggtgctggcttgggtggtggctttggtggtggctttgctggtggtgatgggcttctggtgggcagtgagaaggtgaccatgcagaacctcaatgaccgcctggcctcctacctggacaaggtgcgtgctctggaggaggccaacgccgacctggaagtgaagatccgtgactggtaccagaggcagcggcctgctgagatcaaagactacagtccctacttcaagaccattgaggacctgaggaacaagattctcacagccacagtggacaatgccaatgtccttctgcagattgacaatgcccgtctggccgcggatgacttccgcaccaagtatgagacagagttgaacctgcgcatgagtgtggaagccgacatcaatggcctgcgcagggtgctggacgaactgaccctggccagagctgacctggagatgcagattgagagcctgaaggaggagctggcctacctgaagaagaaccacgaggaggagatgaatgccctgagaggccaggtgggtggagatgtcaatgtggagatggacgctgcacctggcgtggacctgagccgcattctgaacgagatgcgtgaccagtatgagaagatggcagagaagaaccgcaaggatgccgaggaatggttcttcaccaagacagaggagctgaaccgcgaggtggccaccaacagcgagctggtgcagagcggcaagagcgagatctcggagctccggcgcaccatgcagaacctggagattgagctgcagtcccagctcagcatgaaagcatccctggagaacagcctggaggagaccaaaggtcgctactgcatgcagctggcccagatccaggagatgattggcagcgtggaggagcagctggcccagctccgctgcgagatggagcagcagaaccaggagtacaagatcctgctggacgtgaagacgcggctggagcaggagatcgccacctaccgccgcctgctggagggcgaggacgcccacctctcctcctcccagttctcctctggatcgcagtcatccagagatgtgacctcctccagccgccaaatccgcaccaaggtcatggatgtgcacgatggcaaggtggtgtccacccacgagcaggtccttcgcaccaagaactga

SEQ ID NO: 6 - protein, 472 aa, Homo sapiens
MTTCSRQFTSSSSMKGSCGIGGGIGGGSSRISSVLAGGSCRAPSTYGGGLSVSSSRFSSGGAYGLGGGYGGGFSSSSSSFGSGFGGGYGGGLGAGLGGGFGGGFAGGDGLLVGSEKVTMQNLNDRLASYLDKVRALEEANADLEVKIRDWYQRQRPAEIKDYSPYFKTIEDLRNKILTATVDNANVLLQIDNARLAADDFRTKYETELNLRMSVEADINGLRRVLDELTLARADLEMQIESLKEELAYLKKNHEEEMNALRGQVGGDVNVEMDAAPGVDLSRILNEMRDQYEKMAEKNRKDAEEWFFTKTEELNREVATNSELVQSGKSEISELRRTMQNLEIELQSQLSMKASLENSLEETKGRYCMQLAQIQEMIGSVEEQLAQLRCEMEQQNQEYKILLDVKTRLEQEIATYRRLLEGEDAHLSSSQFSSGSQSSRDVTSSSRQIRTKVMDVHDGKVVSTHEQVLRTKN

SEQ ID NO: 7 - DNA, 2043 bp, Homo sapiens
atgaattttgaaacttcacggtgtgccaccctacagtactgccctgacccttacatccagcgtttcgtagaaaccccagctcatttctcttggaaagaaagttattaccgatccaccatgtcccagagcacacagacaaatgaattcctcagtccagaggttttccagcatatctgggattttctggaacagcctatatgttcagttcagcccattgacttgaactttgtggatgaaccatcagaagatggtgcgacaaacaagattgagattagcatggactgtatccgcatgcaggactcggacctgagtgaccccatgtggccacagtacacgaacctggggctcctgaacagcatggaccagcagattcagaacggctcctcgtccaccagtccctataacacagaccacgcgcagaacagcgtcacggcgccctcgccctacgcacagcccagctccaccttcgatgctctctctccatcacccgccatcccctccaacaccgactacccaggcccgcacagtttcgacgtgtccttccagcagtcgagcaccgccaagtcggccacctggacgtattccactgaactgaagaaactctactgccaaattgcaaagacatgccccatccagatcaaggtgatgaccccacctcctcagggagctgttatccgcgccatgcctgtctacaaaaaagctgagcacgtcacggaggtggtgaagcggtgccccaaccatgagctgagccgtgaattcaacgagggacagattgcccctcctagtcatttgattcgagtagaggggaacagccatgcccagtatgtagaagatcccatcacaggaagacagagtgtgctggtaccttatgagccaccccaggttggcactgaattcacgacagtcttgtacaatttcatgtgtaacagcagttgtgttggagggatgaaccgccgtccaattttaatcattgttactctggaaaccagagatgggcaagtcctgggccgacgctgctttgaggcccggatctgtgcttgcccaggaagagacaggaaggcggatgaagatagcatcagaaagcagcaagtttcggacagtacaaagaacggtgatggtacgaagcgcccgtttcgtcagaacacacatggtatccagatgacatccatcaagaaacgaagatccccagatgatgaactgttatacttaccagtgaggggccgtgagacttatgaaatgctgttgaagatcaaagagtccctggaactcatgcagtaccttcctcagcacacaattgaaacgtacaggcaacagcaacagcagcagcaccagcacttacttcagaaacagacctcaatacagtctccatcttcatatggtaacagctccccacctctgaacaaaatgaacagcatgaacaagctgccttctgtgagccagcttatcaaccctcagcagcgcaacgccctcactcctacaaccattcctgatggcatgggagccaacattcccatgatgggcacccacatgccaatggctggagacatgaatggactcagccccacccaggcactccctcccccactctccatgccatccacctcccactgcacacccccacctccgtatcccacagattgcagcattgtcagtttcttagcgaggttgggctgttcatcatgtctggactatttcacgacccaggggctgaccaccatctatcagattgagcattactccatggatgatctggcaagtctgaaaatccctgagcaatttcgacatgcgatctggaagggcatcctggaccaccggcagctccacgaattctcctccccttctcatctcctgcggaccccaagcagtgcctctacagtcagtgtgggctccagtgagacccggggtgagcgtgttattgatgctgtgcgattcaccctccgccagaccatctctttcccaccccgagatgagtggaatgacttcaactttgacatggatgctcgccgcaataagcaacagcgcatcaaagaggagggggagtga

SEQ ID NO: 8 - protein, 680 aa, Homo sapiens
MNFETSRCATLQYCPDPYIQRFVETPAHFSWKESYYRSTMSQSTQTNEFLSPEVFQHIWDFLEQPICSVQPIDLNFVDEPSEDGATNKIEISMDCIRMQDSDLSDPMWPQYTNLGLLNSMDQQIQNGSSSTSPYNTDHAQNSVTAPSPYAQPSSTFDALSPSPAIPSNTDYPGPHSFDVSFQQSSTAKSATWTYSTELKKLYCQIAKTCPIQIKVMTPPPQGAVIRAMPVYKKAEHVTEVVKRCPNHELSREFNEGQIAPPSHLIRVEGNSHAQYVEDPITGRQSVLVPYEPPQVGTEFTTVLYNFMCNSSCVGGMNRRPILIIVTLETRDGQVLGRRCFEARICACPGRDRKADEDSIRKQQVSDSTKNGDGTKRPFRQNTHGIQMTSIKKRRSPDDELLYLPVRGRETYEMLLKIKESLELMQYLPQHTIETYRQQQQQQHQHLLQKQTSIQSPSSYGNSSPPLNKMNSMNKLPSVSQLINPQQRNALTPTTIPDGMGANIPMMGTHMPMAGDMNGLSPTQALPPPLSMPSTSHCTPPPPYPTDCSIVSFLARLGCSSCLDYFTTQGLTTIYQIEHYSMDDLASLKIPEQFRHAIWKGILDHRQLHEFSSPSHLLRTPSSASTVSVGSSETRGERVIDAVRFTLRQTISFPPRDEWNDFNFDMDARRNKQQRIKEEGE